(19) United States
(12) Patent Application Publication
(10) Pub. No.: US 2014/0296740 A1
(43) Pub. Date: Oct. 2, 2014

(54) IMPLANTABLE PRESSURE MEASURING DEVICE

(71) Applicant: AESCULAP AG, TUTTLINGEN (DE)

(72) Inventors: Klaus-Dieter Steinhilper, Tuttlingen (DE); Olaf Hegemann, Tuebingen (DE); Anton Keller, Duerbheim (DE)

(73) Assignee: AESCULAP AG, TUTTLINGEN (DE)

(21) Appl. No.: 14/264,204

(22) Filed: Apr. 29, 2014

Related U.S. Application Data
(63) Continuation of application No. PCT/EP2012/068317, filed on Sep. 18, 2012.

(30) Foreign Application Priority Data
Nov. 11, 2011 (DE) 10 2011 055 284

(51) Int. Cl. A61B 5/03; A61B 5/00

(52) U.S. Cl. CPC A61B 5/031; A61B 5/0031; A61B 5/6868
USPC 600/561

(57) ABSTRACT

In an implantable pressure measuring device, in particular for measuring an intracranial pressure, is provided. In order to simplify production, it is proposed that the pressure measuring device comprises an implant housing and a pressure measuring sensor, which is arranged in the implant housing, with one or more load cell(s). The implant housing has an opening. The pressure measuring sensor is provided with a coating applied directly to the load cell(s). The pressure measuring sensor is arranged in the implant housing in such a way that the opening provides direct access to the load cell(s) for a fluid surrounding the pressure measuring device and therefore the surrounding pressure acts directly on the coated load cell(s) of the pressure measuring sensor. The coating is produced from a para-xylylene-based polymer material.

This application is a continuation of international application number PCT/EP2012/068317 filed on Sep. 18, 2012 and claims the benefit of German application number DE 10 2011 055 284.7 filed on Nov. 11, 2011 which are incorporated herein by reference in their entirety and for all purposes.

### BACKGROUND OF THE INVENTION

The invention relates to an implantable pressure measuring device, in particular for measuring an intracranial pressure, comprising an implant housing and a pressure measuring sensor, which is arranged in the implant housing, with one or more load cell(s). The detection of intracranial pressure has outstanding significance in many neurosurgical interventions. As precise and simple a measurement of the intracranial pressure is important here and this should, in particular, take place with minimum invasion.

One example of a known implantable pressure measuring device of this type is described in WO 2006/117123 A1, in which the pressure measuring sensor is arranged in a rigid housing that is outwardly closed by a thin biocompatible membrane. The pressure-dependent movement of the membrane acts by means of a transmission means, in particular air or a special gas or else a liquid on the pressure measuring sensor or its load cell.

The object of the present invention is to provide a pressure measuring device that is simple to produce.

### SUMMARY OF THE INVENTION

This object is achieved in the pressure measuring device mentioned in the introduction by the features of claim 1.

The simplification in the production of the implantable pressure measuring device according to the invention is produced, in particular, by the pressure measuring sensor or its load cell(s) being provided with a coating, the latter being exposed to a surrounding liquid and the surrounding pressure thus being able to act directly on the load cell(s) of the pressure measuring sensor. An encapsulation of the pressure measuring sensor and, in particular, also the defined introduction of a transmission medium between a membrane and the pressure measuring sensor are thereby dispensed with. On the other hand, the pressure measuring sensor continues to be protected by the mechanically rigid housing. This moreover simplifies a reduction in the dimensions of the device.

It is moreover advantageously achieved that a more precise measurement of the surrounding pressure becomes possible by the direct action of the surrounding pressure owing to the coating on the load cell(s), as this measurement is not influenced by the elasticity of a membrane or the temperature dependency of the elasticity thereof. Compared to the conventional implantable pressure measuring devices, it should be emphasised, in particular, that no dead volume that could impair the performance is present in the pressure measuring device. The transmission losses owing to a transmission means are thus also dispensed with.

Moreover, no hermetically sealed encapsulation of the pressure measuring device is necessary, as an adequate shielding in relation to surrounding fluid is already ensured by means of the aforementioned coating.

The implantable pressure measuring device according to the invention is used, in particular, as a measuring cell for measuring physiological ambient parameters, for example pressure and temperature, and for the telemetric transmission of the detected measured values to an external evaluation unit. The implantable pressure measuring device may, for example, be used as part of a drainage system for so-called cranio-spinal fluid in the case of regulating disorders of the water balance in the brain.

Simple temperature compensation also becomes possible as no gas volume is present, the expansion of which under the influence of temperature would have to be taken into account during a calibration.

Moreover, smaller transmission losses occur as, on the one hand, the rigidity of the membrane and, on the other hand, the transmission through an interposed medium is dispensed with.

Overall, a simpler calibrating routine is produced with the corresponding time advantage and minimisation of the storage size of the sensor system.

Moreover, a test of the manufactured housing for tightness and also a test of the tightness of the membrane closing the housing are dispensed with during production.

The implantable pressure measuring device of the present invention has a coating, which preferably has a layer thickness in the range from about 0.5 to about 20 μm, more preferably from about 0.5 to about 10 μm and most preferably from about 0.5 to about 5 μm.

Although the para-xylylene-based polymer material is known per se as a relatively stiff material, this is used as the coating and is, in particular in the layer thicknesses given above, sufficiently flexible to lower pressure losses to below a critical size.

The para-xylylene-based polymer material is preferably selected from Parylene, in particular Parylene of the types diX C and diX N.

The pressure measuring sensor is preferably configured as a capacitive pressure measuring sensor.

The pressure measuring sensor with the load cell(s) is preferably configured as a microchip, in particular as an ASIC, and allows accordingly small dimensions of the pressure measuring device as a whole. In this case, it may be provided that the pressure measuring sensor comprises a series of a plurality of micro load cells, by means of which a further increase in accuracy is possible in the pressure measurement by an averaging of a plurality of measured values.

The implant housing itself may be manufactured from titanium, ceramic or plastics material, in particular polyetheretherketone (PEEK).

The housing is preferably configured as a housing open on one side.

The shape of the implant housing is preferably hollow cylindrical, the housing also being able to be a housing open on both sides.

The pressure measuring sensor is arranged here with the load cell(s) in the housing preferably set back relative to this opening, so the pressure measuring sensor is protected by the housing wall from mechanical damage.

The pressure measuring sensor is more preferably arranged on a circuit board, which is preferably manufactured from ceramic.

The circuit board is preferably held in the housing with positive engagement and thus substantially free from mechanical stresses, so a measurement, which is uninfluenced overall, of the surrounding pressure of the pressure measuring device is possible.

The pressure measuring device according to the invention is preferably equipped for a telemetric measured value transmission and for this purpose has a transmission unit, which has an operative connection with the pressure measuring sensor. In connection therewith, the transmission unit is preferably arranged on a circuit board, in particular on a common circuit board, on which the pressure measuring sensor itself is also arranged. Particularly preferably, the transmission unit together with the pressure measuring sensor is integrated in a microchip.

More preferably, the pressure measuring device comprises a sensor for a temperature measurement, the temperature sensor optionally having an operative connection with the transmission unit and preferably being arranged on a common circuit board with the pressure sensor and/or the transmission unit. If the pressure measuring device according to the invention comprises a transmission unit and/or a temperature sensor, this unit and/or this sensor is also likewise coated with the polymer material of the coating of the pressure measuring sensor. The temperature sensor, preferably together with the pressure measuring sensor and optionally the transmission unit, can also be integrated in a microchip.

All the measuring data are preferably wirelessly transmitted.

### BRIEF DESCRIPTION OF THE DRAWINGS

This and further advantages of the invention will be described in more detail below with the aid of the drawings, in which, in detail:

FIG. 1 shows an implantable pressure measuring device according to the invention;

FIG. 2 shows the sensor system of the pressure measuring device of FIG. 1 without a housing; and

FIG. 3 shows a pressure measuring sensor of the pressure measuring device of FIG. 1.

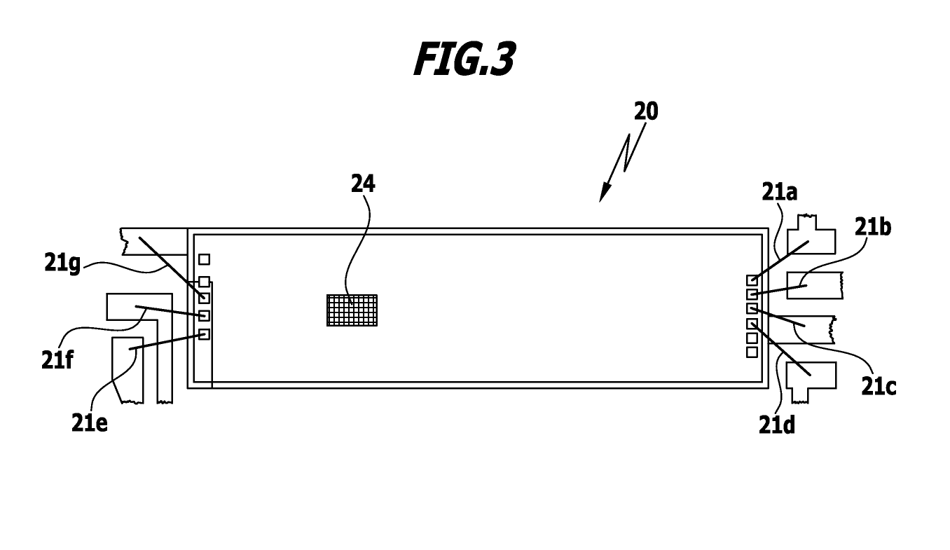

### DETAILED DESCRIPTION

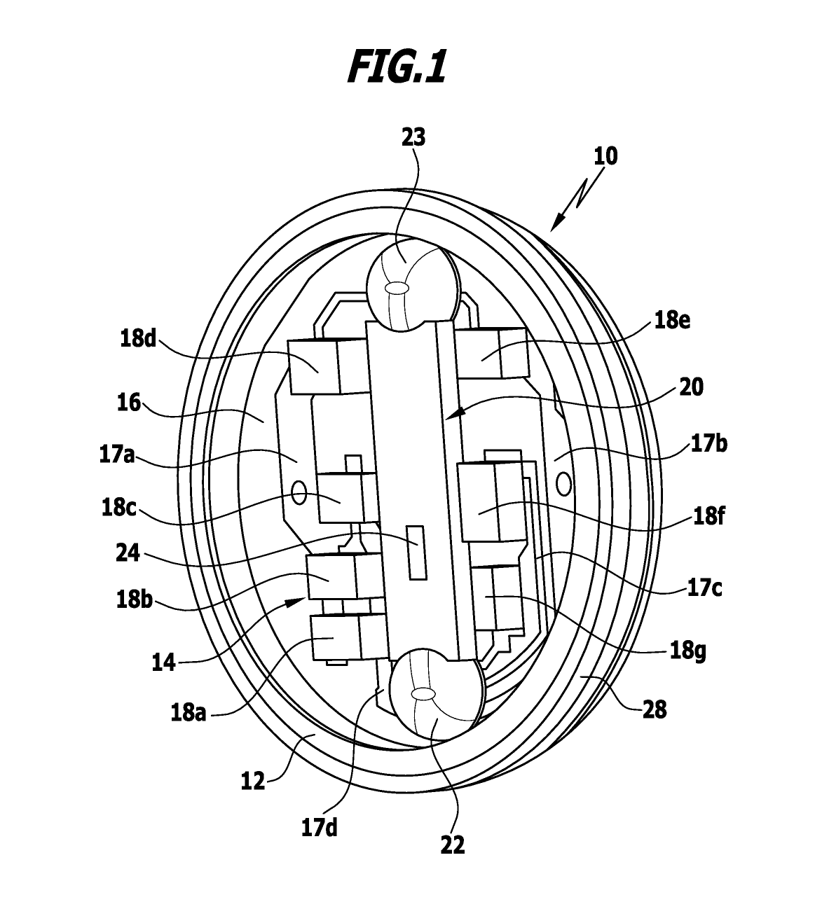

FIG. 1 shows an implantable pressure measuring device according to the invention designated as a whole by the reference numeral 10, with a measuring and transmission unit 16, which is arranged on a board 14. The board 14 is preferably produced from a ceramic material and, apart from the conductor paths 17a to 17d, receives the individual components of the measuring and transmission electronics, i.e. in particular various switching components 18a to 18g to evaluate and transmit the measured pressure values and a pressure measuring sensor 20 configured as an ASIC. The switching components contain, for example, diodes and capacitors.

The connections 21a to 21g of the pressure measuring sensor 20 are protected by means of so-called glob-tops 22, 23.

The entire circuit board 14 and the components assembled thereon including the conductor paths are coated with a Parylene layer, preferably having a thickness of 0.5 to 5 μm.

Parylene is a preferred material from the class of para-xylylene-based polymers and, in particular, apart from its high biocompatibility, has a sufficient protective effect for the electronic components located underneath.

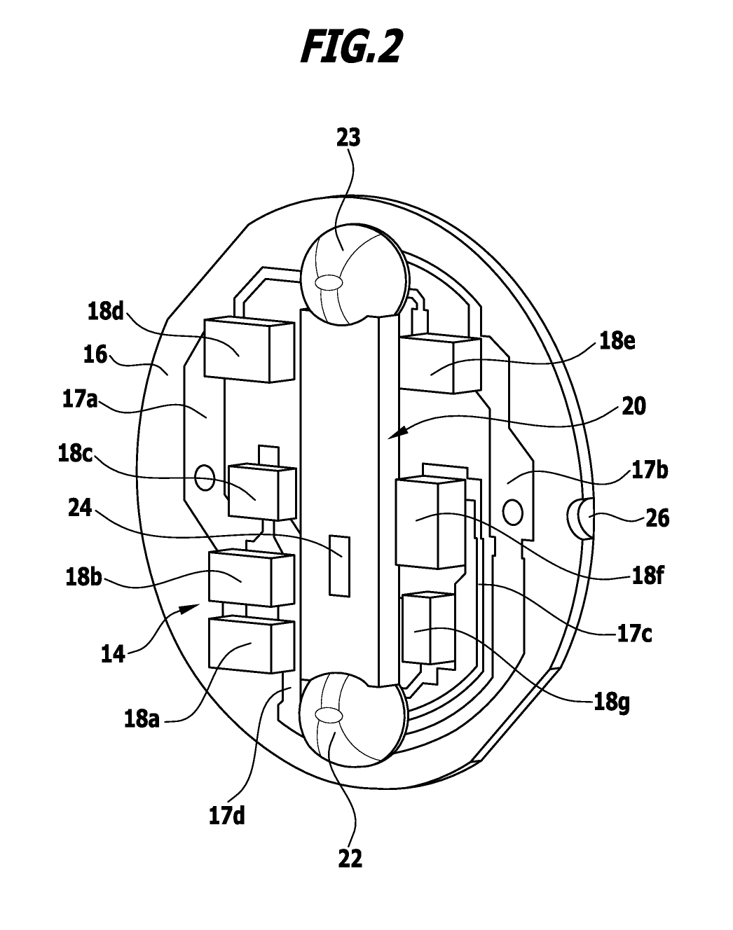

FIG. 2 shows the circuit board 14 without the housing 12, which is preferably closed on one side or else may be configured as a hollow cylindrical part that is open on both sides.

The circuit board 14 with the conductor paths 17a to 17d formed thereon and the assembled components 18a to 18g and 20 is preferably provided with the Parylene layer before assembly in the housing 12.

The circuit board 14 carries, on the rear side, a coil 26, which, on the one hand, is used for energy transmission and, on the other hand, for emitting the telemetric data, which have been detected by the measuring device.

The measuring device of the present invention preferably has a pressure measuring sensor 20, in which the pressure measuring sensor is formed as an array of a plurality of micro load cells, so a multiple measurement of the pressure and an averaging of the measurement result obtained is possible, which leads to a further increase in accuracy.

A temperature sensor is also preferably integrated in the pressure measuring sensor 20 configured as an ASIC, so temperature values, which can be used for a still more accurate evaluation of the measured pressure values can also be transmitted in a pressure measuring device according to the invention. Optionally, the transmission unit is also integrated in the microchip (not shown in detail).

The housing 12 may, for example, be configured as a titanium sleeve or else, more preferably, be produced from ceramic material or plastics material (the latter, in particular PEEK), the non-metallic materials having the advantage that they allow a broader emission characteristic of the implantable pressure measuring device according to the invention.

In the implanted state, the cerebral fluid will come into direct contact with the coating of the circuit board 14 and the components assembled thereon and thus also come into direct contact with the micro load cell array 24. The measurement therefore takes place practically directly, as no transmission medium is present for the pressure and the layer thickness is selected to be sufficiently thin so that no falsification of the measurement results can be adjusted.

The board 14 is preferably held at its edge in the installed state in the housing 12 by means of a positive seating, which excludes mechanical stresses being exerted on the board 14, even if the temperatures of the device should vary in the course of use.

The housing 12 is furthermore configured in such a way that it projects with the edges of its opening beyond the components 18a to 18g and 20 of the circuit board, so they are protected from mechanical influences.

A coating of the board 14 and the structural elements assembled thereon can also take place after fixing in the housing 12, so the circuit board 14 and the components mounted there are preferably then coated together with the housing 12.

The pressure measuring sensor 20 preferably also contains a unit for transmitting the pressure and/or the temperature data determined, which preferably takes place contactlessly or free of body contact by means of the coil 26.

The housing wall is preferably provided on the outside with a radially projecting edge 28, which facilitates the placing of the implantable pressure measuring device according to the invention, for example in a drill hole of a bone.

What is claimed is:

1. Implantable pressure measuring device, in particular for measuring an intracranial pressure, comprising an implant housing and a pressure measuring sensor, which is arranged in the implant housing, with one or more load cell(s), wherein:
the implant housing has an opening,
the pressure measuring sensor is provided with a coating applied directly to the load cell(s),
the pressure measuring sensor is arranged in the implant housing in such a way that the opening provides direct access to the load cell(s) for a fluid surrounding the pressure measuring device and therefore the surrounding pressure acts directly on the coated load cell(s) of the pressure measuring sensor, and
the coating is produced from a para-xylylene-based polymer material.

2. Pressure measuring device according to claim 1, wherein the coating has a layer thickness in the range from about 0.5 to about 20 μm, preferably about 0.5 to about 10 μm, more preferably about 0.5 to about 5 μm.

3. Pressure measuring device according to claim 1, wherein the para-xylylene-based polymer material is selected from Parylene, in particular of the types diX C and diX N.

4. Pressure measuring device according to claim 1, wherein the pressure measuring sensor is a capacitive pressure measuring sensor.

5. Pressure measuring device according to claim 1, wherein the pressure measuring sensor with the load cell(s) is configured as a microchip.

6. Pressure measuring device according to claim 1, wherein the pressure measuring sensor comprises an array of a plurality of micro load cells.

7. Pressure measuring device according to claim 1, wherein the implant housing is manufactured from titanium, ceramic or plastics material, in particular polyetheretherketone (PEEK).

8. Pressure measuring device according to claim 1, wherein the implant housing is hollow cylindrical.

9. Pressure measuring device according to claim 1, wherein the housing is a housing open at one side.

10. Pressure measuring device according to claim 1, wherein the pressure measuring sensor is arranged with the load cell(s) in the housing set back relative to the opening.

11. Pressure measuring device according to claim 1, wherein the pressure measuring sensor is arranged on a circuit board, which is manufactured, in particular, from ceramic.

12. Pressure measuring device according to claim 11, wherein the circuit board is held in the housing with positive engagement substantially free from mechanical stresses.

13. Pressure measuring device according to claim 1, wherein the pressure measuring device comprises a transmission unit, which has an operative connection with the pressure measuring sensor, and the pressure measuring device is, in particular, configured as a telemetric measuring device, the transmission unit preferably being arranged on a circuit board, in particular on a common circuit board with the pressure measuring sensor.

14. Pressure measuring device according to claim 1, wherein the pressure measuring device comprises a sensor for a temperature measurement, the temperature sensor optionally having an operative connection with the transmission unit and preferably being arranged on a common circuit board with the pressure sensor and/or the transmission unit.

15. Pressure measuring device according to claim 11, wherein the coating substantially covers the entire circuit board, the pressure measuring sensor and optionally the transmission unit and/or the temperature sensor.
